SEQUENCE LISTING (34 sequences)

SEQ ID NO: 1 - protein, 951 aa, Artificial Sequence
  ADAMTS-like protein 2
MDGRWQCSCWAWFLLVLAVVAGDTVSTGSTDNSPTSNSLEGGTDATAFWWGEWTKWTACSRSCGGGVTSQERHCLQQRRKSVPGPGNRTCTGTSKRYQLCRVQECPPDGRSFREEQCVSFNSHVYNGRTHQWKPLYPDDYVHISSKPCDLHCTTVDGQRQLMVPARDGTSCKLTDLRGVCVSGKCEPIGCDGVLFSTHTLDKCGICQGDGSSCTHVTGNYRKGNAHLGYSLVTHIPAGARDIQIVERKKSADVLALADEAGYYFFNGNYKVDSPKNFNIAGTVVKYRRPMDVYETGIEYIVAQGPTNQGLNVMVWNQNGKSPSITFEYTLLQPPHESRPQPIYYGFSESAESQGLDGAGLMGFVPHNGSLYGQASSERLGLDNRLFGHPGLDMELGPSQGQETNEVCEQAGGGACEGPPRGKGFRDRNVTGTPLTGDKDDEEVDTHFASQEFFSANAISDQLLGAGSDLKDFTLNETVNSIFAQGAPRSSLAESFFVDYEENEGAGPYLLNGSYLELSSDRVANSSSEAPFPNVSTSLLTSAGNRTHKARTRPKARKQGVSPADMYRWKLSSHEPCSATCTTGVMSAYAMCVRYDGVEVDDSYCDALTRPEPVHEFCAGRECQPRWETSSWSECSRTCGEGYQFRVVRCWKMLSPGFDSSVYSDLCEAAEAVRPEERKTCRNPACGPQWEMSEWSECTAKCGERSVVTRDIRCSEDEKLCDPNTRPVGEKNCTGPPCDRQWTVSDWGPCSGSCGQGRTIRHVYCKTSDGRVVPESQCQMETKPLAIHPCGDKNCPAHWLAQDWERCNTTCGRGVKKRLVLCMELANGKPQTRSGPECGLAKKPPEESTCFERPCFKWYTSPWSECTKTCGVGVRMRDVKCYQGTDIVRGCDPLVKPVGRQACDLQPCPTEPPDDSCQDQPGTNCALAIKVNLCGHWYYSKACCRSCRPPHS

SEQ ID NO: 2 - protein, 1065 aa, Artificial Sequence
  Ceruloplasmin
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 3 - protein, 1231 aa, Artificial Sequence
  Complement factor H sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 4 - protein, 399 aa, Artificial Sequence
  Protein DDI1 homolog2
MLLTVYCVRRDLSEVTFSLQVDADFELHNFRALCELESGIPAAESQIVYAERPLTDNHRSLASYGLKDGDVVILRQKENADPRPPVQFPNLPRIDFSSIAVPGTSSPRQRQPPGTQQSHSSPGEITSSPQGLDNPALLRDMLLANPHELSLLKERNPPLAEALLSGDLEKFSRVLVEQQQDRARREQERIRLFSADPFDLEAQAKIEEDIRQQNIEENMTIAMEEAPESFGQVVMLYINCKVNGHPVKAFVDSGAQMTIMSQACAERCNIMRLVDRRWAGIAKGVGTQKIIGRVHLAQVQIEGDFLPCSFSILEEQPMDMLLGLDMLKRHQCSIDLKKNVLVIGTTGSQTTFLPEGELPECARLAYGAGREDVRPEEIADQELAEALQKSAEDAERQKP

SEQ ID NO: 5 - protein, 313 aa, Artificial Sequence
  Ficolin 2
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 6 - protein, 325 aa, Artificial Sequence
  insulin like growth factor binding protein 2
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 7 - protein, 585 aa, Artificial Sequence
  galectin-3-binding protein
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 8 - protein, 248 aa, Artificial Sequence
  mannose-binding protein C
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 465 aa, Artificial Sequence
  pancreatic triacylglycerol lipase
MLPLWTLSLLLGAVAGKEVCYERLGCFSDDSPWSGITERPLHILPWSPKDVNTRFLLYTNENPNNFQEVAADSSSISGSNFKTNRKTRFIIHGFIDKGEENWLANVCKNLFKVESVNCICVDWKGGSRTGYTQASQNIRIVGAEVAYFVEFLQSAFGYSPSNVHVIGHSLGAHAAGEAGRRTNGTIGRITGLDPAEPCFQGTPELVRLDPSDAKFVDVIHTDGAPIVPNLGFGMSQVVGHLDFFPNGGVEMPGCKKNILSQIVDIDGIWEGTRDFAACNHLRSYKYYTDSIVNPDGFAGFPCASYNVFTANKCFPCPSGGCPQMGHYADRYPGKTNDVGQKFYLDTGDASNFARWRYKVSVTLSGKKVTGHILVSLFGNKGNSKQYEIFKGTLKPDSTHSNEFDSDVDVGDLQMVKFIWYNNVINPTLPRVGASKIIVETNVGKQFNFCSPETVREEVLLTLTPC

SEQ ID NO: 10 - protein, 610 aa, Artificial Sequence
  E-selectin
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 11 - protein, 396 aa, Artificial Sequence
  Sialic acid-binding Ig-like lectin 14
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLTLIRGALMGAGFLLTYGLTWIYYTRCGGPQQSRAERPG

SEQ ID NO: 12 - protein, 1170 aa, Artificial Sequence
  Thrombospondin-1
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 13 - protein, 208 aa, Artificial Sequence
  Zymogen granule protein 16 homolog B
MGAQGAQESIKAMWRVPGTTRRPVTGESPGMHRPEAMLLLLTLALLGGPTWAGKMYGPGGGKYFSTTEDYDHEITGLRVSVGLLLVKSVQVKLGDSWDVKLGALGGNTQEVTLQPGEYITKVFVAFQAFLRGMVMYTSKDRYFYFGKLDGQISSAYPSQEGQVLVGIYGQYQLLGIKSIGFEWNYPLEEPTTEPPVNLTYSANSPVGR

SEQ ID NO: 14 - protein, 10 aa, Artificial Sequence
  ADAMTSL2 peptide
NFNIAGTVVK

SEQ ID NO: 15 - protein, 8 aa, Artificial Sequence
  Cp peptide
GEFYIGSK

SEQ ID NO: 16 - protein, 7 aa, Artificial Sequence
  Cp peptide
AEVGDTI

SEQ ID NO: 17 - protein, 10 aa, Artificial Sequence
  CFH peptide
SLGNIIMVCR

SEQ ID NO: 18 - protein, 10 aa, Artificial Sequence
  CFH peptide
SLGNVIMVCR

SEQ ID NO: 19 - protein, 16 aa, Artificial Sequence
  CFH peptide
CYFPYLENGYNQNYGR

SEQ ID NO: 20 - protein, 16 aa, Artificial Sequence
  CFH peptide
CYFPYLENGYNQNHGR

SEQ ID NO: 21 - protein, 8 aa, Artificial Sequence
  DDI2 peptide
DGDVVILR

SEQ ID NO: 22 - protein, 15 aa, Artificial Sequence
  DDI2 peptide
IDFSSIAVPGTSSPR

SEQ ID NO: 23 - protein, 11 aa, Artificial Sequence
  FCN2 peptide
LQAADTCPEVK

SEQ ID NO: 24 - protein, 9 aa, Artificial Sequence
  IGFBP2 peptide
LIQGAPTIR

SEQ ID NO: 25 - protein, 13 aa, Artificial Sequence
  LGALS3BP peptide
SDLAVPSELALLK

SEQ ID NO: 26 - protein, 8 aa, Artificial Sequence
  MBL2 peptide
WLTFSLGK

SEQ ID NO: 27 - protein, 11 aa, Artificial Sequence
  PNLIP peptide
TGYTQASQNIR

SEQ ID NO: 28 - protein, 11 aa, Artificial Sequence
  PNLIP peptide
GEENWLANVCK

SEQ ID NO: 29 - protein, 13 aa, Artificial Sequence
  PNLIP peptide
VTGHILVSLFGNK

SEQ ID NO: 30 - protein, 10 aa, Artificial Sequence
  SELE peptide
NWAPGEPNNR

SEQ ID NO: 31 - protein, 8 aa, Artificial Sequence
  SELE peptide
QPQNGSVR

SEQ ID NO: 32 - protein, 8 aa, Artificial Sequence
  SIGLEC14 peptide
EGGEFTCR

SEQ ID NO: 33 - protein, 15 aa, Artificial Sequence
  THBS1 peptide
GGVNDNFQGVLQNVR

SEQ ID NO: 34 - protein, 16 aa, Artificial Sequence
  ZG16B peptide
YFSTTEDYDHEITGLR

(19) United States
(12) Patent Application Publication
(10) Pub. No.: US 2022/0146531 A1
(43) Pub. Date: May 12, 2022

(54) A COMBINATION OF BIOMARKERS FOR DIAGNOSING OF DIABETIC RETINOPATHY AND USE THEREOF

(71) Applicant: RETI MARK CO., LTD., Seoul (KR)

(72) Inventors: Seong Jun PARK, Seoul (KR); Young Ju LEE, Gyeonggi-do (KR); Hye Rim KIM, Incheon (KR)

(73) Assignee: RETI MARK CO., LTD., Seoul (KR)

(21) Appl. No.: 17/436,582

(22) PCT Filed: Mar. 6, 2020
(86) PCT No.: PCT/KR2020/003168

§ 371 Date: Sep. 3, 2021

(30) Foreign Application Priority Data
Mar. 7, 2019 (KR) 10-2019-0026302

(51) Int. Cl. G01N 33/68

(52) U.S. Cl. CPC G01N 33/6893; G01N 2800/60; G01N 2800/164

(57) ABSTRACT

The present invention relates to a combination of biomarkers for diagnosing diabetic retinopathy and a use thereof, and more specifically to a combination of biomarkers for diagnosing diabetic retinopathy which is composed of two or more blood biomarkers specific for the diagnosis of diabetic retinopathy with increased diagnostic performance. In addition, the present invention relates to a composition for diagnosing diabetic retinopathy, a diagnostic kit and a method for providing information necessary for the diagnosis of diabetic retinopathy, using the combination of biomarkers. The combination of biomarkers for diagnosing diabetic retinopathy according to the present invention was confirmed to exhibit excellent sensitivity and diagnostic performance compared to other biomarker combinations, and it was also confirmed to exhibit high diagnostic capacity in early diabetic retinopathy when the protein quantitative values of the combination of biomarkers and the basic clinical information were combined and analyzed.

### TECHNICAL FIELD

The present invention relates to a combined biomarker for diagnosing diabetic retinopathy and a use thereof, and more specifically to a combined biomarker for diagnosing diabetic retinopathy which is composed of two or more blood biomarkers specific for the diagnosis of diabetic retinopathy with increased diagnostic performance. In addition, the present invention relates to a composition for diagnosing diabetic retinopathy, a diagnostic kit and a method for providing information necessary for the diagnosis of diabetic retinopathy, using the combined biomarker.

### BACKGROUND ART

Diabetic retinopathy (DR or DMR) is a representative complication of diabetes, which appears when the microvasculature of the retina is damaged, and it is one of the three ophthalmic blindness diseases with age-related macular degeneration and glaucoma. According to the Health Insurance Review and Assessment Service, the number of diabetic patients increased by 21%, from about 2 million in 2012 to about 2.45 million in 2016, but the number of diabetic retinopathy patients increased by 38%, from about 260,000 in 2012 to 336,000 in 2016, and the rate of increase was greater than that of diabetes.

While the blood glucose is increased due to diabetes, the microcirculation of the retinal blood vessels is disturbed due to persistent hyperglycemia, and the blood vessels that supply nutrients to the posterior retina of the eye become narrowed or clogged, and waste products are accumulated, resulting in bleeding. Diabetic retinopathy occurs by abnormally forming new blood vessels at this site, which prevents the normal function of blood vessels and causes bleeding.

Diabetic retinopathy is classified into non-proliferative diabetic retinopathy (NPDR) and proliferative diabetic retinopathy (PDR), depending on the degree of severity. NPDR is also classified as mild, moderate and severe NPDR according to the degree. Non-proliferative diabetic retinopathy shows the manifestations of retinal hemorrhage, microvascular flow, cotton wool spot and the like, but good vision is maintained until the late stage. Proliferative diabetic retinopathy results in the formation of new blood vessels in the retina, and when these blood vessels rupture, it causes severe bleeding in the vitreous cavity, which is absorbed over time, but turns into fibrous tissue, which later causes traction retinal detachment and rebleeding, resulting in permanent blindness.

Diabetic retinopathy has few initial symptoms, making it difficult to diagnose early, and when symptoms (low vision, loss of focus and glare) appear, the disease is already advanced, and in spite of treatment (laser or vitreous surgery), it worsens and leads to blindness in many patients. However, since it is possible to maintain vision if the patient receives proper treatment and thorough blood sugar management at an early stage of the disease onset, the need for early detection and suppression of diabetic retinopathy and early treatment for high-risk groups is emerging. However, the precise etiology has not yet been identified, and there is a problem that biomarkers that determine the progress of retinopathy are very limited.

Accordingly, as a result of attempting to develop a blood protein biomarker having high sensitivity and specificity and using the same in combination to enhance the early diagnostic capacity of diabetic retinopathy, the inventors of the present invention have confirmed that the combined biomarker including mannose-binding protein C (MBL2), pancreatic triacylglycerol lipase (PNLIP), galectin-3-binding protein (LGALS3BP) and insulin-like growth factor binding protein 2 (IGFBP2) has excellent diagnostic efficiency for diabetic retinopathy, and thereby completed the present invention.

### DISCLOSURE

### Technical Problem

An object of the present invention is to provide a combined biomarker for diagnosing diabetic retinopathy, including blood biomarkers specific for the diagnosis of diabetic retinopathy.

Another object of the present invention is to provide a composition or a diagnostic kit for diagnosing diabetic retinopathy using the combined biomarker for diagnosing diabetic retinopathy.

Still another object of the present invention is to provide a method for providing information necessary for the diagnosis of diabetic retinopathy using the combined biomarker for diagnosing diabetic retinopathy.

### Technical Solution

In order to achieve the above objects, the present invention provides a combined biomarker for diagnosing diabetic retinopathy, including mannose-binding protein C (MBL2), pancreatic triacylglycerol lipase (PNLIP), galectin-3-binding protein (LGALS3BP) and insulin-like growth factor binding protein 2 (IGFBP2).

In a preferred exemplary embodiment of the present invention, the combined biomarker may further include one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

In addition, the present invention provides a composition for diagnosing diabetic retinopathy, including an agent for measuring the mRNA or protein level of a combined biomarker for diagnosing diabetic retinopathy, which includes mannose-binding protein C (MBL2), pancreatic triacylglycerol lipase (PNLIP), galectin-3-binding protein (LGALS3BP) and insulin-like growth factor binding protein 2 (IGFBP2).

In a preferred exemplary embodiment of the present invention, the combined biomarker may further include one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

In another preferred exemplary embodiment of the present invention, the agent for measuring the mRNA level of the combined biomarker may be a primer pair, probe or antisense nucleotide that specifically binds to a gene of each biomarker.

In still another preferred exemplary embodiment of the present invention, the agent for measuring the protein level of the combined biomarker may include an antibody, interacting protein, ligand, nanoparticle or aptamer that specifically binds to a protein or peptide fragment of each biomarker.

In addition, the present invention provides a kit for diagnosing diabetic retinopathy, including the composition for diagnosing diabetic retinopathy.

In a preferred exemplary embodiment of the present invention, the kit may be a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit.

In addition, the present invention provides a method for providing information for diagnosing diabetic retinopathy, including (a) measuring the mRNA or protein level of the combined biomarker for diagnosing diabetic retinopathy, including mannose-binding protein C (MBL2), pancreatic triacylglycerol lipase (PNLIP), galectin-3-binding protein (LGALS3BP) and insulin-like growth factor binding protein 2 (IGFBP2), from a biological sample of a patient; and (b) comparing the mRNA or protein expression level with an mRNA or protein expression level from a sample of a control group.

In a preferred exemplary embodiment of the present invention, the combined biomarker may further include one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

In another preferred exemplary embodiment of the present invention, the method for providing information for diagnosing diabetic retinopathy compares with the control group by further including one or more types of clinical information selected from the group consisting of the patient's age, body mass index (BMI), smoking status, Hb1Ac test result, insulin treatment, hypertension, hyperlipidemia and cardiovascular disease.

In still another exemplary embodiment of the present invention, the method for providing information for diagnosing diabetic retinopathy may further include diagnosing diabetic retinopathy if the gene expression level or protein expression level of the combined biomarker increases compared to the control group.

In still another exemplary embodiment of the present invention, the measurement of the mRNA expression level may be performed by using a reverse transcription polymerase chain reaction, a competitive reverse transcription polymerase chain reaction, a real-time reverse transcription polymerase chain reaction, an RNase protection assay, Northern blotting or a DNA chip.

In still another exemplary embodiment of the present invention, the measurement of the protein expression level may be performed by using multiple reaction monitoring (MRM), parallel reaction monitoring (PRM), sequential windowed data independent acquisition of the total high-resolution (SWATH), selected reaction monitoring (SRM) or immune-multiple reaction monitoring (iMRM).

In still another exemplary embodiment of the present invention, the analysis of step (b) may be performed by a statistical analysis method.

In still another exemplary embodiment of the present invention, the statistical analysis method may be selected from the group consisting of a linear or non-linear regression analysis method, a linear or non-linear classification analysis method, a logistic regression analysis method, an analysis of variance (ANOVA), a neural network analysis method, a genetic analysis method, a support vector machine analysis method, a hierarchical analysis or clustering analysis method, a hierarchical algorithm or kernel principal component analysis method using decision tree, the Markov Blanket analysis method, a recursive feature elimination or entropy-based regression feature elimination analysis method, a forward floating search or backward floating search analysis method, and a combination thereof.

### Advantageous Effects

The combined biomarker for diagnosing diabetic retinopathy according to the present invention was confirmed to exhibit excellent sensitivity and diagnostic performance compared to other biomarker combinations, and it was also confirmed to exhibit high diagnostic capacity in early diabetic retinopathy when the protein quantitative values of the combined biomarker and the basic clinical information were combined and analyzed. In addition, the combined biomarker of the present invention can be effectively used for the early diagnosis of diabetic retinopathy because it can be conveniently analyzed using the patient plasma without using a biopsy as a blood protein.

### DESCRIPTION OF DRAWINGS

FIG. 1 is data obtained by combining the protein quantification values of a combined biomarker and the basic clinical information of all diabetic retinopathy patients, and analyzed by statistical processing with a logistic regression model (A) and T-test (B) (DMR: diabetic retinopathy, Non DMR: non-diabetic retinopathy).

FIG. 2 is data obtained by combining the protein quantification values of a combined biomarker and the basic clinical information of patients with non-proliferative diabetic retinopathy, and analyzed by statistical processing with a logistic regression model (A) and T-test (B) (NPDR: non-proliferative diabetic retinopathy, Non DMR: non-diabetic retinopathy).

FIG. 3 is data obtained by combining the protein quantification values of a combined biomarker and the basic clinical information of patients with proliferative diabetic retinopathy, and analyzed by statistical processing with a logistic regression model (A) and T-test (B) (PDR: proliferative diabetic retinopathy, Non DMR: non-diabetic retinopathy).

FIG. 4 is data obtained by combining the protein quantitative values of a combined biomarker and the basic clinical information of patients with non-proliferative diabetic retinopathy at each stage, and analyzed by statistical processing with a T-test.

A is data comparing non-diabetic retinopathy (Non DMR) and mild non-proliferative diabetic retinopathy (mild NPDR), B is data comparing non-diabetic retinopathy (Non DMR) and moderate diabetic retinopathy (moderate NPDR), and C is data comparing non-diabetic retinopathy (Non DMR) and severe diabetic retinopathy (severe NPDR).

FIG. 5 is data obtained by combining the protein quantitative values according to a combination of MLB2, IGFBP2, LGALS3BP and PNLIP (Combination 1) and a combination of ADAMTSL2, CP, DDI, FCN2, SIGLEC14, SELE, THBS1, ZG16B and CFH (Combination 2) among the 13 biomarkers identified in the present invention and the basic clinical information of all diabetic retinopathy patients, and analyzed by statistical processing with a logistic regression model.

### MODES OF THE INVENTION

Hereinafter, the present invention will be described in detail.

In one aspect, the present invention relates to a combined biomarker for diagnosing diabetic retinopathy, including mannose-binding protein (MBL2), pancreatic triacylglycerol lipase (PNLIP), gelatin-3-binding proteins (LGALS3BP) and insulin-like growth factor binding protein (IGFBP2).

As used herein, the term “diagnosis” means identifying the presence or characteristics of a pathological condition. For the purposes of the present invention, the diagnosis is to determine whether diabetic retinopathy has occurred.

As used herein, the term “diagnostic biomarker” includes organic biomolecules such as polypeptides or nucleic acids (e.g., mRNA, etc.), lipids, glycolipids, glycoproteins, sugars (monosaccharides, disaccharides, oligosaccharides, etc.) and the like, which show a significantly increased or decreased pattern of the gene expression level or protein expression level in a subject with diabetic retinopathy as compared to a normal control group (a subject with no diabetic retinopathy).

Depending on the degree of progression, diabetic retinopathy is classified into early non-proliferative diabetic retinopathy (NPDR) and late proliferative diabetic retinopathy (PDR). Non-proliferative diabetic retinopathy is characterized by no vascular development. Proliferative diabetic retinopathy differs in the mechanism such as the development of blood vessels, and non-proliferative diabetic retinopathy does not necessarily progress to proliferative diabetic retinopathy, and even a biomarker known as a biomarker for the diagnosis of proliferative diabetic retinopathy may not necessarily be used as a biomarker for the diagnosis of non-proliferative diabetic retinopathy.

The combined biomarker of the present invention may specifically diagnose both non-proliferative diabetic retinopathy and proliferative diabetic retinopathy, and may particularly diagnose early stages of non-proliferative diabetic retinopathy.

In the present invention, the combined biomarker may further include one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

ADAMTS-like protein 2 (ADAMTSL2) is a member of the ADAMTS (a disintegrin and metalloproteinase with thrombospondin motifs)-like protein subfamily and is a glycoprotein that binds to the cell surface and the extracellular matrix. ADAMTSL2 interacts with LTBP1 (latent transforming growth factor beta binding protein 1), and since the LTBP1 protein is involved in the storage of TGF-β1, which is an important growth factor that regulates the growth and division of cells, ADAMTSL2 regulates the availability of TGF-β1. In addition, ADAMTSL2 is expressed in cancer tissues and therefore used as a biomarker for cancer diagnosis.

In the present invention, the ADAMTSL2 may preferably include the amino acid sequence of SEQ ID NO: 1, or may include a sequence that is at least 90%, at least 93%, at least 95%, at least 96%, at least 97%, at least 98%, or at least 99% identical to the amino acid sequence of SEQ ID NO: 1.

Ceruloplasmin (Cp) plays an important role in iron metabolism as a major protein carrying copper in blood. At least about 95% of copper present in the plasma of healthy persons is in the form of ceruloplasmin.

In the present invention, the Cp may preferably include the amino acid sequence of SEQ ID NO: 2, or may include a sequence that is at least 90%, at least 93%, at least 95%, at least 96, at least 97%, at least 98%, or at least 99% identical to the amino acid sequence of SEQ ID NO: 3.

Complement factor H (CFH) is a glycoprotein that plays an essential role in regulating complement activation to maintain an immune response. It serves as a complement inhibitor that binds to the same sugar chain structure on the cell surface and prevents complement activation and amplification.

In the present invention, the CFH may preferably include the amino acid sequence of SEQ ID NO: 3, or may include a sequence that is at least 90%, at least 93%, at least 95%, at least 96, at least 97%, at least 98%, or at least 99% identical to the amino acid sequence of SEQ ID NO: 3.

Protein DDI1 homolog 2 (DD12) is an internal peptide cleaving enzyme that activates nuclear respiratory factor 1 (Nrf1), which is involved in the regulation of cell growth and DNA replication, to complement protein degradation caused by abnormalities in the proteasome.

In the present invention, the DDI2 may preferably include the amino acid sequence of SEQ ID NO: 4, or may include a sequence that is at least 90%, at least 93%, at least 95%, at least 96%, at least 97%, at least 98%, or at least 99% identical to the amino acid sequence of SEQ ID NO: 4.

Ficolin 2(FCN2) is a type of oligolectin and is composed of a short N-terminal part-collagen-like domain and a fibrinogen-like domain. It is mainly expressed in the liver and is known to play an important role in the lectin pathway of the complement system by binding to N-acetylglucosamine in the bacterial cell wall and acting as an opsonin like mannose-binding protein.

In the present invention, the FCN2 may preferably include the amino acid sequence of SEQ ID NO: 5, or may include a sequence that is at least 90%, at least 93%, at least 95%, at least 96%, at least 97%, at least 98%, or at least 99% identical to the amino acid sequence of SEQ ID NO: 5.

Insulin-like growth factor binding protein 2 (IGFBP2) binds to insulin-like growth factor (IGF) and modulates a variety of processes in the cell and thereby regulates angiogenesis. In addition, it is known to promote the growth of cancer cells in various cancers (prostate cancer, breast cancer, etc.).

In the present invention, the IGFBP2 may preferably include the amino acid sequence of SEQ ID NO: 6, or may include a sequence that is at least 90%, at least 93%, at least 95%, at least 96%, at least 97%, at least 98%, or at least 99% identical to the amino acid sequence of SEQ ID NO: 6.

Galectin-3-binding protein (LGALS3BP) is a protein encoded by the LGALS3BP gene and specifically binds to human macrophage-associated lectin (Mac-2) and galectin 1. LGALS3BP is known to be increased in the serum of cancer patients and HIV-infected patients and is involved in immune responses associated with the cytotoxicity of natural killer (NK) cells and lymphokine-activated killer (LAK) cells.

In the present invention, the LGALS3B may preferably include the amino acid sequence of SEQ ID NO: 7, or may include a sequence that is at least 90%, at least 93%, at least 95%, at least 96%, at least 97%, at least 98%, or at least 99% identical to the amino acid sequence of SEQ ID NO: 7.

Mannose-binding protein C (MBL2) is also referred to as mannose-binding lectin (MBL) or mannan-binding protein (MBP). MBL2 has an oligomeric structure (400 to 700 kDa) and is composed of subunits containing three identical peptide chains composed of approximately 30 kDa. It is produced by the liver in response to infection and is part of many other factors called acute-phase proteins.

In the present invention, the MBL2 may preferably include the amino acid sequence of SEQ ID NO: 8, or may include a sequence that is at least 90%, at least 93%, at least 95%, at least 96%, at least 97%, at least 98%, or at least 99% identical to the amino acid sequence of SEQ ID NO: 8.

Pancreatic triglyceride lipase (PNLIP) is a group of lipolytic enzymes that hydrolyze the ester bonds of triglycerides and is an enzyme secreted in the pancreas. Although PNLIP is known to be low in serum concentration because it is secreted into the duodenum through the pancreas duct system, it is known that when pancreatic functions are extremely disrupted by pancreatitis or pancreatic adenocarcinoma, pancreatic enzymes including PNLIP are secreted into serum, and thus, the PNLIP serum concentration may be measured to diagnose acute pancreatitis.

In the present invention, the PNLIP may preferably include the amino acid sequence of SEQ ID NO: 9, or may include a sequence that is at least 90%, at least 93%, at least 95%, at least 96%, at least 97%, at least 98%, or at least 99% identical to the amino acid sequence of SEQ ID NO:9.

E-selectin (SELE) is known as CD62 antigen-like family member E (CD62E), endothelial-leukocyte adhesion molecule 1 (ELAM) or leukocyte-endothelial cell adhesion molecule 2 (LECAM2), and it is a cell adhesion molecule that is transiently expressed and induced in vascular endothelial cells activated by interleukin 1β, tumor necrosis factor and lipopolysaccharide, peaking at 4 to 12 hours. SELE is strongly expressed in vascular endothelial cells in the inflamed tissue and mediates the rolling phenomenon of neutrophils and monocytes on vascular endothelial cells, thereby promoting infiltration of these cells into the inflammatory site. It is also known to be involved in the adhesion of cancer cells to vascular endothelial cells.

In the present invention, the SELE may preferably include the amino acid sequence of SEQ ID NO: 10, or may include a sequence that is at least 90%, at least 93%, at least 95%, at least 96%, at least 97%, at least 98%, or at least 99% identical to the amino acid sequence of SEQ ID NO: 10.

Sialic acid-binding Ig-like lectin 14 (SIGLEC14) is one of the subfamilies of sialic acid-binding immunoglobulin-type lectins (SIGLEC), and SIGLEC is a cell surface protein that binds to sialic acid and is mainly expressed on the surface of immune cells. The protein interaction between SIGLEC and sialic acid acts as a switch that turns the immune system on and off, and it is known that cancer cells also acquire resistance to the immune response using the SIGLEC-sialic acid reaction.

In the present invention, the SIGLEC14 may preferably include the amino acid sequence of SEQ ID NO: 11, or may include a sequence that is at least 90%, at least 93%, at least 95%, at least 96%, at least 97%, at least 98%, or at least 99% identical to the amino acid sequence of SEQ ID NO: 11.

Thrombospondin-1 (THBS1) is one of the thrombospondin families, and it is a glycoprotein that inhibits angiogenesis and tumorigenesis. It is known that it binds to proteases associated with angiogenesis such as plasminogen, urokinase, MMP, thrombin, cathepsin and the like, in order to regulate the adhesion, migration and growth of endothelial cells.

In the present invention, the THBS1 may preferably include the amino acid sequence of SEQ ID NO: 12, or may include a sequence that is at least 90%, at least 93%, at least 95%, at least 96%, at least 97%, at least 98%, or at least 99% identical to the amino acid sequence of SEQ ID NO: 12.

Zymogen granule protein 16 homolog B (ZG16B) is a pancreatic adenocarcinoma upregulated factor (PAUF), and it is known to bind to carbohydrates and activate the angiogenesis and permeability of endothelial cells.

In the present invention, the ZG16B may preferably include the amino acid sequence of SEQ ID NO: 13, or may include a sequence that is at least 900%, at least 93%, at least 95%, at least 96%, least 97%, at least 98%, or at least 99% identical to the amino acid sequence of SEQ ID NO: 13.

However, there is no known prior art that discloses that the above biomarkers may be used for the diagnosis of diabetic retinopathy, and in particular, there is no known technique that discloses that the combined biomarker of MBL2, PNLIP, LGALS3BP and IGFBP2 may be used to increase the diagnostic performance of diabetic retinopathy.

In another aspect, the present invention relates to a composition for diagnosing diabetic retinopathy, including an agent for measuring the mRNA or protein level of a combined biomarker for diagnosing diabetic retinopathy, which includes mannose-binding protein C (MBL2), pancreatic triacylglycerol lipase (PNLIP), galectin-3-binding protein (LGALS3BP) and insulin-like growth factor binding protein 2 (IGFBP2).

The combined biomarker may be characterized by further including one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

In the present invention, the agent for measuring the mRNA level of the combined biomarker is characterized to be a primer pair, a probe or an antisense nucleotide that specifically binds to the genes of the biomarkers, and since the nucleic acid information of the genes is known in GeneBank and the like, a person skilled in the art may design such a primer pair, probe or antisense nucleotide based on the sequence.

As used herein, the term “measurement of mRNA expression level” is to measure the amount of mRNA in a biological sample isolated from a patient suspected of diabetic retinopathy in order to diagnose the diabetic retinopathy by determining the presence of mRNA and the degree of expression of genes for diagnosing diabetic retinopathy.

As used herein, the term “primer” is a fragment that recognizes a target gene sequence and includes forward and reverse primer pairs, and preferably, it is a primer pair that provides assay results with specificity and sensitivity. High specificity may be imparted when the nucleic acid sequence of the primer is a sequence that is mismatched with the non-target sequence present in the sample such that only the target gene sequence containing a complementary primer binding site is amplified and the primer does not cause non-specific amplification.

As used herein, the term “probe” means a substance capable of specifically binding to a target substance to be detected in a sample, and means a substance capable of specifically confirming the presence of the target substance in the sample through the binding. The type of probe is not limited as a material commonly used in the art, but may preferably be peptide nucleic acid (PNA), locked nucleic acid (LNA), peptide, polypeptide, protein, RNA or DNA, and most preferably PNA. More specifically, the probe includes those derived from or similar to an organism as a biomaterial or those prepared ex vivo, and may be, for example, an enzyme, protein, antibody, microorganism, animal and plant cells and organs, neuron, DNA, and RNA. In addition, DNA may include cDNA, genomic DNA and oligonucleotides, RNA may include genomic RNA, mRNA and oligonucleotide, and examples of proteins may include antibodies, antigens, enzymes, peptides and the like.

As used herein, the term “antisense” refers to an oligomer having a sequence of nucleotide bases and an inter-subunit backbone such that the antisense oligomer hybridizes to a target sequence within the RNA by the Watson-Crick base pairing, thereby allowing the formation of mRNA and RNA:oligomer heterodimers typically within the target sequence. The oligomer may have exact sequence complementarity or approximate complementarity to a target sequence.

In the present invention, the agent for measuring the protein level of the combined biomarker may be an antibody, interacting protein, ligand, nanoparticle or aptamer that specifically binds to the protein or peptide fragment.

As used herein, the term “measurement of protein expression level” is a process for determining the presence and the degree of expression of proteins expressed from genes for diagnosing diabetic retinopathy in a biological sample in order to diagnose diabetic retinopathy.

As used herein, the term “antibody” refers to a substance that specifically binds to an antigen and causes an antigen-antibody reaction. For the purposes of the present invention, an antibody means an antibody which binds specifically to the combined biomarker for diagnosing diabetic retinopathy according to the present invention. The antibodies of the present invention include all of polyclonal antibodies, monoclonal antibodies and recombinant antibodies. The antibodies may be easily prepared using techniques well known in the art. For example, the polyclonal antibodies may be produced by a method well known in the art, including the process of injecting the protein antigen of the biomarker for diabetic retinopathy into an animal and collecting blood from the animal to obtain a serum containing the antibody. Such polyclonal antibodies may be prepared from any animal such as a goat, rabbit, sheep, monkey, horse, pig, cow, dog and the like. In addition, monoclonal antibodies may be prepared using the hybridoma method well known in the art (refer to Kohler and Milstein, European Journal of Immunology 6:511-519, 1976), or the phage antibody library technique (refer to Clackson et al., Nature, 352:624-628, 1991; Marks et al., J. Mol. Biol., 222:58, 1-597, 1991). The antibody produced by the above method may be isolated and purified using methods such as gel electrophoresis, dialysis, salt precipitation, ion exchange chromatography, affinity chromatography and the like. In addition, the antibodies of the present invention include functional fragments of antibody molecules as well as complete forms having two full-length light chains and two full-length heavy chains. A functional fragment of an antibody molecule means a fragment having at least an antigen-binding function, and examples thereof include Fab. F(ab′), F(ab′)2, Fv and the like. The antibodies of the present invention may also be commercially obtained.

As used herein, the term “peptide nucleic acid (PNA)” refers to an artificially synthesized DNA or RNA-like polymer. While DNA has a phosphate-ribose backbone, PNA has a repeated N-(2-aminoethyl)-glycine backbone linked by peptide bonds, which greatly increases the binding capacity and stability to DNA or RNA, and thus, it is used in molecular biology, diagnostic assays and antisense therapies. PNA is described in detail in the document (Nielsen P E et al., Science, 254(5037):1497-5), 1991).

In the present invention, an “aptamer” is an oligonucleotide or peptide molecule, and the general content of aptamers is described in detail in the documents (Bock L C et al., Nature, 355(6360):5646, 1992; Hoppe-Seyler F and Butz K. J Mol Med., 78(8):42630, 2000; Cohen B A et al., Proc Natl Acad Sci USA., 95(24):142727, 1998).

In another aspect, the present invention relates to a kit for diagnosing diabetic retinopathy, including the composition for diagnosing diabetic retinopathy.

The kit may be prepared by the conventional manufacturing methods known in the art. The kit may include, for example, an antibody in lyophilized form and a buffer, a stabilizer, an inert protein and the like.

The kit may further include a detectable label. The term “detectable label” refers to an atom or molecule that specifically detects a molecule including a label among the same type of molecules without the label. The detectable label may be one attached to an antibody, interacting protein, ligand, nanoparticle, or aptamer that specifically binds to the protein or fragment thereof. The detectable label may include a radionuclide, a fluorophore or an enzyme.

The kit may be used according to various immunoassays or immunostaining methods known in the art. The immunoassays or immunostaining may include radioimmunoassay, radioimmunoprecipitation, immunoprecipitation. ELISA, capture-ELISA, inhibition or competition assays, sandwich assays, flow cytometry, immunofluorescence and immunoaffinity purification. Preferably, the kit may be a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit.

In addition, the kit may be used for mass spectrometry. In this case, certain amino acid residues of the protein may have modifications such as myristoylation, isoprenylation, prenylation, glypication, lipoylation, acylation, alkylation, methylation, demethylation, amidation, ubiquitination, phosphorylation, deamidation, glycosylation, oxidation, or acetylation and the like.

In another aspect, the present invention relates to a method for providing information for diagnosing diabetic retinopathy, including the steps of (a) measuring the mRNA or protein level of a combined biomarker for diagnosing retinopathy comprising mannose-binding protein C (MBL2), pancreatic triacylglycerol lipase (PNLIP), galectin-3-binding protein (LGALS3BP) and insulin-like growth factor binding protein 2 (IGFBP2) from a biological sample of a patient; and (b) comparing the mRNA or protein expression level with an mRNA or protein expression level from a sample of a control group.

In the present invention, the combined biomarker of step (a) further includes one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

In the above method, the “biological sample” means a tissue, cell, blood, serum, plasma, saliva, cerebrospinal fluid, urine or the like, whose protein expression level or gene expression level differs by the onset of diabetic retinopathy, and it preferably means blood, plasma or serum.

The method for providing information for diagnosing diabetic retinopathy may further include one or more types of clinical information selected from the group consisting of the patient's age, body mass index (BMI), smoking status, Hb1Ac test result, insulin treatment, hypertension, hyperlipidemia and cardiovascular disease.

In addition, the method for providing information for diagnosing diabetic retinopathy may further include the step of diagnosing diabetic retinopathy if the gene expression level or protein expression level of the combined biomarker increases compared to the control group.

The measurement of the mRNA expression level in step (a) may be measured and compared using a primer pair, a probe or an antisense nucleotide that specifically binds to the gene of the combined biomarker.

As the method for measuring or comparing the mRNA expression level, the reverse transcription polymerase chain reaction, competitive reverse transcription polymerase chain reaction, real-time reverse transcription polymerase chain reaction, RNase protection assay. Northern blotting, DNA chip and the like may be used, but the present invention is not limited thereto. The above-described measurement methods may confirm the mRNA expression level of a normal control group and the mRNA expression level of a diabetic retinopathy patient, and by comparing these expression levels, it is possible to diagnose or predict whether the onset of diabetic retinopathy has occurred.

The measurement of the protein expression level in step (a) may be measured and compared using antibodies, interacting proteins, ligands, nanoparticles or aptamers that specifically bind to a protein or peptide fragment.

Examples of the method for measuring or comparing the protein expression level include the protein chip analysis, immunoassay, ligand binding assays, MALDI-TOF (matrix desorption/ionization time-of-flight mass spectrometry) analysis, SELDITOF (surface enhanced laser desorption or ionization time-of-fight mass spectrometry) analysis, radioimmunoassay, radioimmunodiffusion, Ouchterlony immunodiffusion, Rocket immunoelectrophoresis, tissue-immunostaining, complement fixation assay, two-dimensional electrophoresis analysis, liquid chromatography-mass spectrometry (LC-MS), liquid chromatography-mass spectrometry/mass spectrometry (LCMS/MS), Western blot, enzyme-linked immunosorbent assay (ELISA) and the like, but are not limited thereto More preferably, in the present invention, it may be measured using multiple reaction monitoring (MRM), parallel reaction monitoring (PRM), sequential windowed data dependent acquisition of the total high-resolution (SWATH), selected reaction monitoring (SRM) or immune-multiple reaction monitoring (iMRM).

The MRM is a method of determining the exact fragment of a substance and breaking it in a mass spectrometer, and then selecting a specific ion from the broken ions once more to obtain the number using a continuously connected detector. When the MRM method is used, the corresponding protein or a fragment thereof may be quantified using a mass spectrometer in blood samples of a normal subject and a subject suspected of diabetic retinopathy.

The analysis of step (b) may be performed using a statistical method or algorithm to improve the accuracy of the diagnosis, and may use an analysis method selected from the group consisting of a linear or non-linear regression analysis method, a linear or non-linear classification analysis method, a logistic regression analysis method, an analysis of variance (ANOVA), a neural network analysis method, a genetic analysis method, a support vector machine analysis method, a hierarchical analysis or clustering analysis method, a hierarchical algorithm or kernel principal component analysis method using decision tree, the Markov Blanket analysis method, a recursive feature elimination or entropy-based regression feature elimination analysis method, a forward floating search or backward floating search analysis method, and combinations thereof. Preferably in the present invention, the statistical method used a logistic regression analysis method, but is not limited thereto.

In one specific embodiment of the present invention, 155 plasma samples were analyzed for qualitative validation of biomarkers for the diagnosis of diabetic retinopathy in plasma proteomes (Table 1), and 13 biomarkers consisting of insulin-like growth factor binding protein 2 (IGFBP2), ADAMTS-like protein 2 (ADAMTSL2), complement factor H (CFH), ceruloplasmin (Cp), protein DDI1 homolog 2 (DDI2), ficolin 2 (FCN2), galectin 3 binding protein (LGALS3BP), mannose-binding protein C (MBL2), pancreatic triacylglycerol lipase (PNLIP), E-selectin (SELE), sialic acid-binding Ig-like lectin 14 (SIGLEC14), thrombospodin-1 (THBS1) and zymogen granule protein 16 homolog B (ZG16B) were analyzed using multiple reaction monitoring (MRM). As shown in Table 2, MRM-MS analysis was performed using SIS by selecting peptides for each biomarker for quantitative analysis.

In another specific embodiment of the present invention, when the results of the combined biomarker group and the clinical information were combined, the probability value of being classified as diabetic retinopathy was estimated by inputting the conversion information of the biomarker expression level and the degree of conversion of the clinical information using a logistic regression model in order to confirm the improvement effect of diagnostic performance. As shown in Table 3, based on clinical information+MBL2+PNLIP+LGALS3BP+IGFBP2, the expression levels for ADAMTSL2, Cp, DDI2, FCN2, SELE, SIGLEC14, THBS1, ZG16B and CFH were added one by one and analyzed, and as a result, as the number of the biomarkers increased, it was confirmed that the diagnostic capacity of diabetic retinopathy increased.

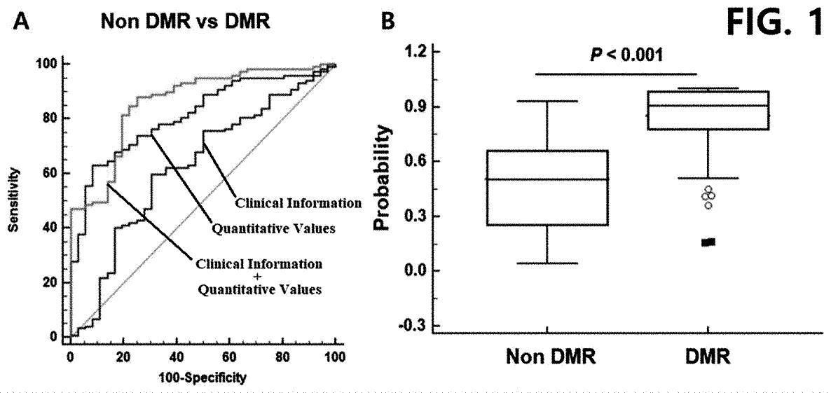

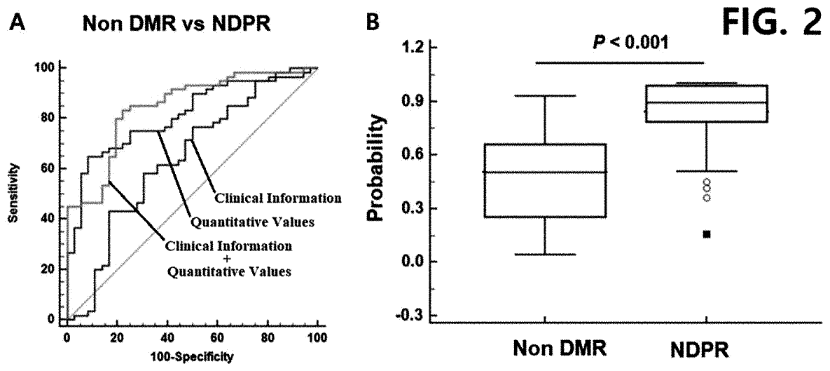

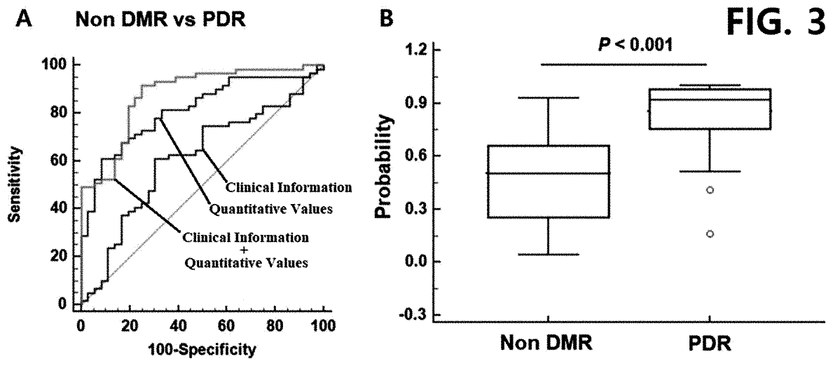

In addition, as shown in FIG. 1 to FIG. 3 and Table 4, when the protein quantitative values for the above-mentioned 13 biomarkers and the basic clinical information were combined and statistically analyzed using the logistic regression model, it was confirmed that the diagnostic capacities for the normal group and diabetic retinopathy (STAGE 1, AUC=0.863), the normal group and non-proliferative diabetic retinopathy (STAGE 2, AUC=0.848), and the normal group and proliferative diabetic retinopathy (STAGE 3, AUC=0.879) were excellent.

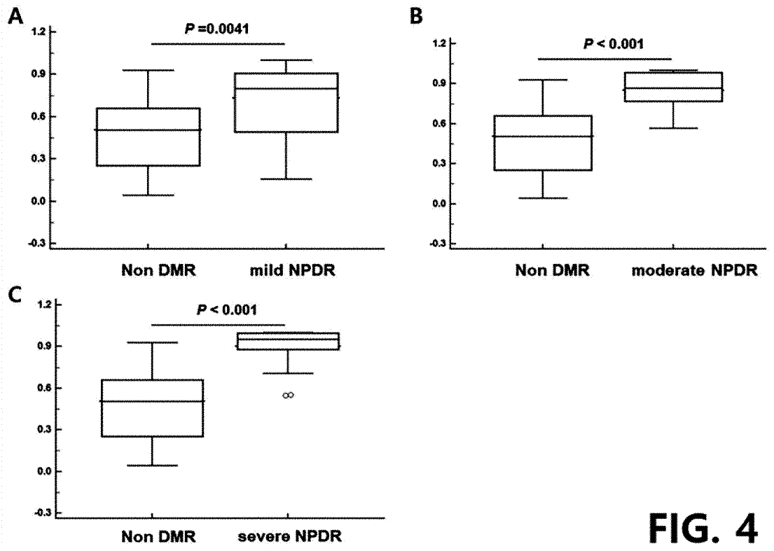

In addition, as shown in FIG. 4, through the T-test, it was confirmed that at various stages of non-proliferative diabetic retinopathy, the diagnostic capacity at each stage was excellent compared to the normal group.

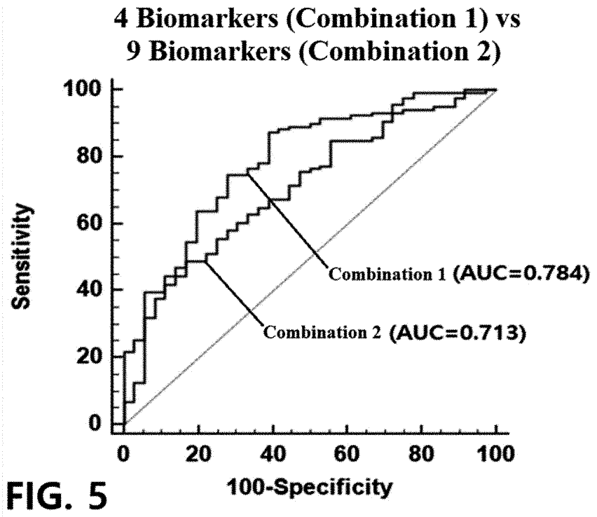

In another specific embodiment of the invention, it was determined whether a difference in diagnostic performance was observed depending on the combination of biomarkers. As shown in FIG. 5, it was confirmed that the combination of MBL2. PNLIP, LGALS3BP and IGFPB2 (Combination 1, AUC=0.783) had higher diagnostic capacity than the combination of the 9 other proteins, ADAMTSL2, Cp, FCN2 DDI2, SELE, SIGLEC14, THBS1 ZG16B and CFH (Combination 2, AUC=0.713) among the 13 targets.

Hereinafter, the present invention will be described in more detail through examples.

These examples are only for illustrating the present invention, and it will be apparent to those of ordinary skill in the art that the scope of the present invention is not to be construed as being limited by these examples.

### Example 1

Selection and Plasma Collection of Patients with Diabetic Retinopathy

Plasma samples from patients with diabetic retinopathy were obtained with approval by the Clinical Trial Review Committee of Seoul National University Bundang Hospital. A total of 155 plasma samples were analyzed for quantitative detection of biomarkers using plasma proteomes, and the clinical characteristics of the normal group (Non DMR) and the diabetic retinopathy (DMR) disease group to be analyzed are shown in Table 1 below.

TABLE 1
Clinical information of samples

| | Sample (n = 155) | | | | | |
|---|---|---|---|---|---|---|
| | | | NPDR (n = 60) | | | |
| | Non DMR | DMR | Mild | Moderate | Severe | PDR |
| | (n = 36) | (n = 119) | (n = 17) | (n = 17) | (n = 26) | (n = 59) |
| Age | 64.1 ± 8.3 | 57.5 ± 9.5 | 57.4 ± 10.9 | 58.9 ± 12.1 | 60.0 ± 7.6 | 56.1 ± 9.0 |
| Gender | 29/7 | 29/90 | 12/5 | 3/14 | 5/21 | 9/50 |
| (female/male) | | | | | | |
| Hb1Ac | 7.4 ± 1.3 | 7.6 ± 1.4 | 7.5 ± 0.9 | 7.6 ± 1.4 | 7.5 ± 1.4 | 7.6 ± 1.5 |
| Diabetic treatment | 32 (88.9) | 116 (97.5) | 17 (100.0) | 17 (100.0) | 25 (96.2) | 57 (96.6) |
| (%) | | | | | | |

Systemic risk factor

| | | | | | | |
|---|---|---|---|---|---|---|
| BMI (%) | | | | | | |
| Low | 4 (11.1) | 7 (5.9) | 0 (0) | 1 (5.9) | 3 (11.5) | 3 (5.1) |
| Normal | 19 (52.8) | 64 (53.8) | 8 (47.1) | 8 (47.1) | 13 (50.0) | 35 (59.3) |
| Moderate | 12 (33.3) | 37 (31.1) | 8 (47.1) | 4 (23.5) | 9 (34.6) | 15 (25.4) |
| High | 1 (2.8) | 11 (9.2) | 1 (5.9) | 4 (23.5) | 0 (0) | 6 (10.2) |
| Smoking (%) | 12 (33.3) | 53 (43.7) | 10 (58.5) | 8 (47.1) | 10 (38.5) | 24 (40.7) |
| Hypertension (%) | 19 (52.8) | 65 (54.6) | 6 (35.3) | 10 (58.8) | 14 (53.8) | 35 (59.3) |
| Hyperlipidemia (%) | 25 (69.4) | 50 (42.0) | 9 (52.9) | 8 (47.1) | 10 (38.5) | 23 (39.0) |
| Cardiovascular | 5 (13.9) | 9 (7.6) | 1 (5.9) | 1 (5.9) | 2 (7.7) | 5 (8.5) |
| disease (%) | | | | | | |

### Example 21

Peptide Selection and Peptide Analysis Using MRM-MS

2-1: Peptide Selection and Synthetic Peptide Design

In the present invention, as biomarkers for diagnosing diabetic retinopathy, 13 biomarkers of insulin-like growth factor binding protein 2 (IGFBP2), ADAMTS-like protein 2 (ADAMTSL2), complement factor H (CFH), ceruloplasmin (Cp), protein DDI1 homolog 2 (DDI2), ficolin 2 (FCN2), galectin 3 binding protein (LGALS3BP), mannose-binding protein C (MBL2), pancreatic triacylglycerol lipase (PNLIP), E-selectin (SELE), sialic acid-binding Ig-like lectin 14 (SIGLEC14), thrombospodin-1 (THBS1) and zymogen granule protein 16 homolog B (ZG16B) were selected.

In order to perform MRM analysis on the biomarkers, a representative peptide having a specific charge-to-mass ratio (m/z) for the proteins of the 13 biomarkers was selected (Qi), and among the fragmentation ions generated by breaking this peptide with an electric shock, the ion (Q3) with the highest strength was selected.

One or more peptides with high sensitivity per protein were measured and injected into a mass spectrometer based thereon to obtain the optimal fragmentation energy value for each transition, and three or more top fragmentation ions were selected based on the strength (Table 2).

TABLE 2
Top fragmentation ions for 13 biomarkers

| Name of gene | Trypsin fragment | SEQ ID NO. | Target ion | Target m/z |
|---|---|---|---|---|
| ADAMTSL2 | NFNIAGTVVK | SEQ ID NO: 14 | +2y6 | 531.801/574.356 |
| Cp | GEFYIGSK | SEQ ID NO: 15 | +2y6 | 450.728/714.382 |
| | AEVGDTI | SEQ ID NO: 16 | +2y5 | 430.727/561.299 |
| CFH | SLGNIIMVCR | SEQ ID NO: 17 | +2y5 | 581.807/678.343 |
| | SLGNVIMVCR | SEQ ID NO: 18 | +2y5 | 574.799/678.343 |
| | CYFPYLENGYNQNYGR | SEQ ID NO: 19 | +2y14 + 2 | 1029.444/867.897 |
| | CYFPYLENGYNQNHGR | SEQ ID NO: 20 | +3y13 + 2 | 677.964/781.361 |
| DDI2 | DGDVVILR | SEQ ID NO: 21 | +2y4 | 443.753/500.355 |
| | IDFSSIAVPGTSSPR | SEQ ID NO: 22 | +2y7 | 767.399/701.358 |
| FCN2 | LQAADTCPEVK | SEQ ID NO: 23 | +2y8 | 616.303/919.419 |
| IGFBP2 | LIQGAPTIR | SEQ ID NO: 24 | +2y6 | 484.798/614.362 |
| LGALS3BP | SDLAVPSELALLK | SEQ ID NO: 25 | +2y8 | 678.393/870.529 |
| MBL2 | WLTFSLGK | SEQ ID NO: 26 | +2y6 | 476.269/652.366 |
| PNLIP | TGYTQASQNIR | SEQ ID NO: 27 | +2y6 | 619.81/688.374 |
| | GEENWLANVCK | SEQ ID NO: 28 | +2y5 | 660.306/591.292 |
| | VTGHILVSLFGNK | SEQ ID NO: 29 | +3y4 | 462.270/465.246 |
| SELE | NWAPGEPNNR | SEQ ID NO: 30 | +2y7 | 577.771/783.374 |
| | QPQNGSVR | SEQ ID NO: 31 | +2y6 | 443.230/660.342 |
| SIGLEC14 | EGGEFTCR | SEQ ID NO: 32 | +2y3 | 478.201/436.197 |
| THBS1 | GGVNDNFQGVLQNVR | SEQ ID NO: 33 | +2y7 | 808.911/785.463 |
| ZG16B | YFSTTEDYDHEITGLR | SEQ ID NO: 34 | +3y7 | 649.63/825.458 |

Stable-isotope labeled standard (SIS) peptides corresponding to 13 proteins were used to confirm quantification, and the spiked heavy peptide concentration was determined using an experiment to confirm linearity via calibration. The SIS peptide is a peptide in which 12C and 14N at the C-terminal lysine (Lys, K) or arginine (Arg, R) amino acid of the peptide are substituted with 13C and 15N. It differs in mass from endogenous peptides present in the blood, but since it has the same sequence, the peptide hydrophobicity is identical, and thus, it elutes at the same retention time (RT) on a chromatogram.

2-2: Pretreatment of Plasma Samples and MRM-MS Analysis

Each plasma obtained in Example 1 above was used as it was, or for more accurate protein quantification, depletion was performed to remove 14 proteins (albumin, IgG, antitrypsin, IgA, transferrin, haptoglobin, fibrinogen, alpha 2-macroglobulin, alpha 1-acid glycoprotein, IgM, apolipoprotein AI, apolipoprotein AII, complement C3 and transthyretin) present in high amounts. For depletion, the multiple affinity removal system (MARS, Agilent, USA) column was used according to the manufacturer's protocol to remove the 14 proteins and the remaining proteins were eluted in a small amount and used for analysis. Urea was added to the eluted depletion sample to 8 M, and then 2-carboxyethyltriphosphine (tris(2-carboxyethyl)-phosphine, TECP) was added to 80 mM and 2-chloroacetamide was added to 320 mM, respectively, and it was reacted at 25° C. for 1 hour to reduce alkylate and disulfide bonds. LysC enzyme was added thereto such that the mass ratio of the plasma protein and the LysC (wako) enzyme was 100:1, and it was reacted at room temperature for 4 hours. Afterwards, it was diluted 10-fold with a 50 mM Tris (pH 8.0) solution and trypsin (Promega) was added at a sequencing-able level such that the mass ratio of the protein and trypsin became 50:1, and it was reacted at 37° C. for 12 hours or more and digested into peptide fragments. A formic acid solution was added to be 0.3% to acidify at pH of 2.0 or less, and the trypsin reaction was stopped.

Herein, desalination was carried out, and a heavy-labeled peptide was added (spiking) as an internal standard material (SIS peptide) determined in Example 2-1, and MRM analysis was performed. It was coupled to Nano ultra 2D plus (Eksigent) and was monitored in the scheduled MRM mode for the transition of each selected protein using the QTarp 5500 (SCIEX) equipment, which is a triple quadrupole mass spectrometer.

Specifically, an ion voltage of 5,500 volts was used, and the resolution at Quadruple 1 (Qi) and Quadruple 3 (Q3) was set in units. The transition was set such that the total cycle time was 1.5 seconds. 20 units of nebulizing gas were used and the heater temperature was set at 350° C. In order to correct for batch-to-batch variation, each sample was spiked with an internal standard material and monitored simultaneously.

2-3: Data Analysis

Raw data was processed using Skyline (McCoss lab, University of Washington, USA) to calculate the peak area of the transition. Relative concentrations were compared using peak areas for the endogenous/heavy labeled peptide. In order to measure the predictive ability of each protein peptide on disease using the measured result values, T-test and AUROC (area under the receiver operating characteristic) values were generated, and in order to confirm the predictive ability combined with the results of the combined biomarker groups and the clinical information, the conversion information of the expression levels and the degree of conversion of the clinical information of Table 1 were inputted using a logistic regression model to estimate the probability values that were classified as diabetic retinopathy. All statistical analyses were performed using MedCal ver. 17.1 (MedCalc).

The clinical information reflected the body mass index (BMI) calculated by the height and body weight, smoking status, the presence of hyperlipidemia, hypertension and cardiovascular disease, and hemoglobin (Hb1Ac) levels obtained through questionnaire. Among these, the information used for the presence or absence was converted to Yes=1 and No=0 (stop smoking=0.5) and reflected.

### Example 31

Confirmation of Improvement in Diagnostic Capacity by Combining Results of Combined Biomarker Groups and Clinical Information

In the present invention, in order to confirm the improvement effect of the diagnostic capacity when the results of the 13 combined biomarkers and the clinical information were combined, the probability value of being classified as diabetic retinopathy was estimated by inputting the conversion information of the biomarker expression levels and the degree of conversion of the clinical information using a logistic regression model.

TABLE 3
Confirmation of diagnostic performance of diabetic retinopathy according to
the combination number of combined biomarkers

| C1 (Combination 1) | | | | | C2 | C3 | C4 | C5 | C6 | C7 | C8 | C9 | C10 | AUC |
|---|---|---|---|---|---|---|---|---|---|---|---|---|---|---|
| 1 | 2 | 3 | 4 | 5 | | | | | | | | | | 0.784 |
| 1 | 2 | 3 | 4 | 5 | 6 | | | | | | | | | 0.803 |
| 1 | 2 | 3 | 4 | 5 | 6 | 7 | | | | | | | | 0.804 |
| 1 | 2 | 3 | 4 | 5 | 6 | 7 | 8 | | | | | | | 0.812 |
| 1 | 2 | 3 | 4 | 5 | 6 | 7 | 8 | 9 | | | | | | 0.814 |
| 1 | 2 | 3 | 4 | 5 | 6 | 7 | 8 | 9 | 10 | | | | | 0.819 |
| 1 | 2 | 3 | 4 | 5 | 6 | 7 | 8 | 9 | 10 | 11 | | | | 0.823 |
| 1 | 2 | 3 | 4 | 5 | 6 | 7 | 8 | 9 | 10 | 11 | 12 | | | 0.824 |
| 1 | 2 | 3 | 4 | 5 | 6 | 7 | 8 | 9 | 10 | 11 | 12 | 13 | | 0.834 |
| 1 | 2 | 3 | 4 | 5 | 6 | 7 | 8 | 9 | 10 | 11 | 12 | 13 | 14 | 0.863 |

1. Clinical Information,
2. LGALS3BP,
3. MBL2,
4. IGFBP2,
5. PNLIP,
6. ZG16B,
7. DDI2,
8. FCN2,
9. THBS1,
10. SIGLEC14,
11. CP,
12. ADAMTSL2,
13. SELC,
14. CFH

TABLE 4
Confirmation of diagnostic capacity according to the combination of
combined biomarker groups and clinical information

| | Non DMR vs DMR | | | Non DMR vs NPDR | | | Non DMR vs PDR | | |
|---|---|---|---|---|---|---|---|---|---|
| | (STAGE 1) | | | (STAGE 2) | | | (STAGE 3) | | |
| | AUC | SE | 95% CI | AUC | SE | 95% CI | AUC | SE | 95% CI |
| C | 0.628 | 0.0550 | 0.520 to | 0.644 | 0.0611 | 0.524 to | 0.611 | 0.0602 | 0.493 to |
| | | | 0.735 | | | 0.764 | | | 0.729 |
| P | 0.813 | 0.0368 | 0.741 to | 0.814 | 0.0434 | 0.729 to | 0.813 | 0.0439 | 0.727 to |
| | | | 0.886 | | | 0.900 | | | 0.899 |
| Com | 0.863 | 0.0342 | 0.796 to | 0.848 | 0.0401 | 0.769 to | 0.879 | 0.0362 | 0.808 to |
| | | | 0.930 | | | 0.927 | | | 0.949 |

C: Clinical information,
P: Protein quantitative information,
Com: Combination of clinical information and protein quantitative information,
AUC: Area Under the Curve,
SE: Standard Error (Delong et al., 1988), 95%
CI: 95% Confidence Interval

As shown in Table 3, based on clinical information+MBL2+PNLIP+LGALS3BP+IGFBP2, the expression levels for ADAMTSL2, Cp, FCN2, DDI2, SELE, SIGLEC14, THBS1, ZG16B and CFH were added one by one and analyzed, and as a result, as the number of the biomarkers increased, it was confirmed that the diagnostic capacity of diabetic retinopathy increased.

In addition, as shown in FIG. 1 to FIG. 3 and Table 4, when the protein quantitative values for the above-mentioned 13 biomarkers and the basic clinical information were combined and statistically analyzed using the logistic regression model, it was confirmed that the diagnostic capacities for the normal group and diabetic retinopathy (STAGE 1. AUC=0.863), the normal group and non-proliferative diabetic retinopathy (STAGE 2, AUC=0.848), and the normal group and proliferative diabetic retinopathy (STAGE 3, AUC=0.879) were excellent.

In addition, as shown in FIG. 4, through the T-test, it was confirmed that at various stages of non-proliferative diabetic retinopathy, the diagnostic capacity at each stage was excellent compared to the normal group.

### Example 4

Confirmation of Diagnostic Performance of Diabetic Retinopathy According to the Combination of Biomarkers

In the present invention, it was confirmed whether there was a difference in the diagnostic performances of diabetic retinopathy according to the combinations of the 13 biomarkers. The 13 biomarkers were divided into a combination of MBL2, PNLIP. LGALS3BP and IGFBP2 and a combination of ADAMTSL2, Cp, FCN2, DDI2, SELE, SIGLEC14, THBS1, ZG16B and CFH to perform analysis, and the analysis was performed together with clinical information in the same manner as in STAGE 1 of Example 3.

As shown in FIG. 5, it was confirmed that the combination of MBL2, PNLIP, LGALS3BP and IGFPB2 (Combination 1, AUC=0.784) had higher diagnostic capacity than the combination of the 9 other proteins, ADAMTSL2, Cp, FCN2 DDI2, SELE, SIGLEC14, THBS1 ZG16B and CFH (Combination 2, AUC=0.713) among the 13 biomarkers.

### INDUSTRIAL APPLICABILITY

The combined biomarker for diagnosing diabetic retinopathy according to the present invention was confirmed to exhibit excellent sensitivity and diagnostic performance compared to other biomarker combinations, and it was also confirmed to exhibit high diagnostic capacity in early diabetic retinopathy when the protein quantitative values of the combined biomarker and the basic clinical information were combined and analyzed.

1. A combination of biomarkers for diagnosing diabetic retinopathy, comprising mannose-binding protein C (MBL2), pancreatic triacylglycerol lipase (PNLIP), galectin-3-binding protein (LGALS3BP) and insulin-like growth factor binding protein 2 (IGFBP2).

2. The combination of biomarkers of claim 1, wherein the combination of biomarkers further comprises one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

3. A composition for diagnosing diabetic retinopathy, comprising a plurality of agent for measuring the mRNA or protein level of a combination of biomarkers for diagnosing diabetic retinopathy, the combination of biomarkers comprising mannose-binding protein C (MBL2), pancreatic triacylglycerol lipase (PNLIP), galectin-3-binding protein (LGALS3BP) and insulin-like growth factor binding protein 2 (IGFBP2).

4. The composition of claim 3, wherein the composition further comprises an agent for measuring the mRNA or protein level of one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

5. The composition of claim 3, wherein one or more of the plurality of agent for measuring the mRNA level of the combination of biomarkers is a primer pair, a probe or an antisense nucleotide that specifically binds to a gene of a biomarker of the combination of biomarkers.

6. The composition of claim 3, wherein one or more of the plurality of agents for measuring the protein level of the combination of biomarkers is an antibody, an interacting protein, a ligand, a nanoparticle or an aptamer that specifically binds to a protein or peptide fragment of a biomarker of the combination of biomarkers.

7. A kit for diagnosing diabetic retinopathy, comprising the composition according to claim 3.

8. The kit of claim 7, wherein the kit is a reverse transcription polymerase chain reaction (RT-PCR) kit, a DNA chip kit, an enzyme-linked immunosorbent assay (ELISA) kit, a protein chip kit, a rapid kit or a multiple reaction monitoring (MRM) kit.

9. A method for diagnosing diabetic retinopathy, comprising:
(a) measuring the mRNA or protein level of the combination of biomarkers of claim 1 from a biological sample of a patient; and
(b) comparing the mRNA or protein expression level with an mRNA or protein expression level from a sample of a control group.

10. The method of claim 9, wherein the method further comprises comparing one or more types of clinical information selected from the group consisting of the patient's age, body mass index (BMI), smoking status, Hb1Ac test result, insulin treatment, hypertension, hyperlipidemia and cardiovascular disease, with the same clinical information from the control group.

11. The method of claim 9, wherein the method further comprises (c) diagnosing diabetic retinopathy if the mRNA or protein expression level of the combination of biomarkers is increased compared to the control group.

12. The method of claim 9, wherein the mRNA level of one or more biomarkers of the combination of biomarkers is measured using a primer pair, a probe or an antisense nucleotide that specifically binds to a gene of a biomarker of the combination of biomarkers.

13. The method of claim 9, wherein the protein level of one or more biomarkers of the combination of biomarkers is measured using an antibody, an interacting protein, a ligand, a nanoparticle or an aptamer that specifically binds to a protein or peptide fragment of a biomarker of the combination of biomarkers.

14. The method of claim 9, wherein the combination of biomarkers further comprises one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).

15. A method for preparing a biological sample for assessing diabetic retinopathy, comprising:
(a) preparing an amplification product or a detection product of the mRNA or protein of a combination of biomarkers from a biological sample of a patient, the combination of biomarkers comprising mannose-binding protein C (MBL2), pancreatic triacylglycerol lipase (PNLIP), galectin-3-binding protein (LGALS3BP) and insulin-like growth factor binding protein 2 (IGFBP2); and
(b) analyzing the amplification product or the detection product by comparing the level of the amplification product or the detection product to the level of an amplification product or a detection product of the same mRNA or protein from a sample of a control group.

16. The method of claim 15, wherein the method further comprises comparing one or more types of clinical information selected from the group consisting of the patient's age, body mass index (BMI), smoking status, Hb1Ac test result, insulin treatment, hypertension, hyperlipidemia and cardiovascular disease, with the same clinical information from the control group.

17. The method of claim 15, wherein the method further comprises (c) diagnosing diabetic retinopathy if the level of the amplification product or the detection product of the combination of biomarkers is increased compared to the control group.

18. The method of claim 15, wherein the level of the amplification product or the detection product of the combination of biomarkers is measured using a primer pair, a probe or an antisense nucleotide that specifically binds to a gene of a biomarker of the combination of biomarkers.

19. The method of claim 15, wherein the level of the detection product or the detection product of the combination of biomarkers is measured using an antibody, an interacting protein, a ligand, a nanoparticle or an aptamer that specifically binds to a protein or peptide fragment of a biomarker of the combination of biomarkers.

20. The method of claim 15, wherein the combination of biomarkers further comprises one or more biomarkers selected from the group consisting of ADAMTS-like protein 2 (ADAMTSL2), ceruloplasmin (Cp), complement factor H (CFH), protein DDI1 homolog 2 (DDI2), ficolin 2 (FCN2), E-selectin (SELE), sialic acid-binding Ig-like lectin 14 (SIGLEC14), thrombospondin-1 (THBS1) and zymogen granule protein 16 homolog B (ZG16B).
